(19) United States
(12) Patent Application Publication
(10) Pub. No.: US 2025/0369960 A1
(43) Pub. Date: Dec. 4, 2025

(54) PER- AND POLYFLUORINATED ALKYL COMPOUND (PFAS) BINDING PROTEINS AND USES THEREOF

(71) Applicant: Allonnia, LLC, Boston, MA (US)

(72) Inventors: Kent Sorenson, Hingham, MA (US); Areen Banerjee, Arlington, MA (US); Dayal Saran, Melrose, MA (US)

(21) Appl. No.: 18/878,050

(22) PCT Filed: Jun. 30, 2023
(86) PCT No.: PCT/US2023/026722

§ 371 Date: Dec. 22, 2024

Related U.S. Application Data
(60) Provisional application No. 63/357,267, filed on Jun. 30, 2022.
(60) Provisional application No. 63/470,583, filed on Jun. 2, 2023.

(51) Int. Cl. G01N 33/53; C07K 16/44

(52) U.S. Cl. CPC G01N 33/5308; C07K 16/44

(57) ABSTRACT

Provided herein are per- and polyfluoroalkyl compound (PFAS) binding proteins. including anti-PFAS antibodies and biosimilars thereof, and uses of the compositions for detecting PFAS.

### FIELD OF THE INVENTION

The invention relates to per-and polyfluoroalkyl compound (PFAS) binding proteins, including anti-PFAS antibodies, including nanobodies, monobodies, single chain variable fragments, and biosimilars thereof, uses of the compositions for detecting PFAS, and devices containing PFAS binding proteins for detecting PFAS.

### BACKGROUND OF THE INVENTION

Per-and polyfluoroalkyl compounds (PFAS) are a class of substances that have been used in industrial and commercial manufacturing over the last six decades. Some properties that have made them attractive to the automotive, healthcare, energy, and storage industries are their chemical inertness, thermal resistance, and protection from weather and abrasion. The extreme stability of these compounds owing to the C—F bond, which is considered the toughest chemical bond to break, has meant that these compounds are now ubiquitous in the environment. In fact, past surveys have found detectable levels of PFAS in serum of 97% of individuals. PFAS has been linked to cancers, elevated cholesterol, obesity, and suppression and disruption of the immune and endocrine systems, respectively. The US EPA PFAS Action Plan, released in February 2019, indicated that the agency would establish a revised drinking water maximum contaminant level (MCL) in line with recent studies that have been performed on the deleterious effects of PFAS on human health and the environment. Existing market estimates place the size of the environmental liability in the US at over $80 billion. Current solutions for PFAS remediation are focused on sequestration techniques via adsorption media and then transfer to a landfill or incineration facility. A big gap in the remediation effort is the rapid detection of ultra-low concentrations of PFAS. The ideal scenario for field detection would be a portable chip-based unit that can detect single digit parts-per-trillion (ppt) levels of PFAS compounds with minimum operator actions within 1.5 h of sampling. Another important factor in this detection system would be to have specificity of binding to PFAS compounds and avoid detection of an interfering moiety like octanoic acid.

It is hypothesized that development of an antibodies (from various animals) that can bind specifically to perfluorooctanoic acid (PFOA), which is one of the most regulated PFAS compounds, would allow detection of very low concentrations of PFOA in water matrix. There is a need in the art for improved compositions for detecting PFAS.

### SUMMARY OF THE INVENTION

Provided herein is a composition comprising a per-and polyfluoroalkyl compound (PFAS) binding protein. The PFAS binding protein may bind to one or more PFAS compounds (that is, may have PFAS compound binding activity). The PFAS compounds may comprise one or more straight chain perfluorinated carboxylic acid molecules comprising a chain of 4 to 10 carbons in length. The PFAS compounds may comprise one or more straight chain perfluorinated sulfonic acid molecules comprising a chain of 4 to 10 carbons in length. The PFAS compounds may comprise at least one of perfluorobutanoic acid (PFBA), perfluoropentanoic acid (PFPeA), perfluorohexanoic acid (PFHxA), perfluoroheptanoic acid (PFHpA), perfluorooctanoic acid (PFOA), perfluorononanoic acid (PFNA), perfluorodecanoic acid (PFDA), perfluorobutane sulfonate (PFBS), perfluoropentane sulfonic acid (PFPeS), perfluorohexanesulphonic acid (PFHxS), perfluoroheptanesulfonic acid (PfHpS), perfluorooctane sulfonic acid (PFOS), perfluorononanesulfonic acid (PFNS), and perfluorodecane sulfonic acid (PFDS). The PFAS compounds may comprise one or more of PFBA, PFPeA, PFHxA, PFHpA, PFOA, PFNA, and PFDA. The PFAS compounds may comprise PFOA, PFOS, PFHxS, PFBA, or a combination thereof. The PFAS compounds may comprise PFOA.

The PFAS binding protein may comprise an anti-PFAS antibody, a biosimilar thereof, or an antigen binding fragment of the foregoing. The anti-PFAS antibody may be a heterotetrameric antibody or a single domain antibody. The heterotetrameric antibody may be produced in a chicken, mouse, rat, rabbit, guinea pig, sheep, pig, goat, or horse. The heterotetrameric antibody may be polyclonal or monoclonal. The anti-PFAS antibody may be a single domain antibody, which may be a nanobody or a single chain variable fragment (scFv). The nanobody may be produced in a camelid, which may be an alpaca, llama, or camel. The PFAS binding protein may be bound to at least one of the PFAS compounds.

The anti-PFAS antibody may comprise a heavy chain variable region and a light chain variable region. The heavy chain variable region may comprise the sequence set forth in one of SEQ ID NOs: 15-19. The light chain variable region may comprise the sequence set forth in one of SEQ ID NOs: 20-24. The heavy chain variable region may comprise the sequence set forth in SEQ ID NO: 15 and the light chain variable region may comprise the sequence set forth in SEQ ID NO: 20. The heavy chain variable region may comprise the sequence set forth in SEQ ID NO: 16 and the light chain variable region may comprise the sequence set forth in SEQ ID NO: 21. The heavy chain variable region may comprise the sequence set forth in SEQ ID NO: 17 and the light chain variable region may comprise the sequence set forth in SEQ ID NO: 22. The heavy chain variable region may comprise the sequence set forth in SEQ ID NO: 18 and the light chain variable region may comprise the sequence set forth in SEQ ID NO: 23. The heavy chain variable region may comprise the sequence set forth in SEQ ID NO: 19 and the light chain variable region may comprise the sequence set forth in SEQ ID NO: 24. The antibody may comprise mAB1, mAB2, mAB3, mAB4, or mAB5, or a biosimilar thereof.

Provided herein is a PFAS detection system comprising the composition. The PFAS binding protein may be in a solution or attached to a solid substrate. The solid substrate may comprise an electroactive surface. The solid substrate may comprise an optical waveguide surface. The optical waveguide surface may comprise a channel. The solid substrate may comprise a metal. The metal may comprise gold. The solid substrate may comprise at least one functional molecule that binds the PFAS binding protein, which may be an anti-PFAS antibody. A cartridge may comprise the solid substrate.

Provided herein is a method of detecting a PFAS compound in a sample. The method may comprise contacting the sample with the composition or the detection system under conditions that allow the PFAS binding protein to bind the PFAS compound, resulting in a PFAS binding protein-PFAS compound conjugate. The method may comprise detecting the level of the PFAS binding protein-PFAS compound conjugate. The level of the PFAS binding protein-PFAS compound conjugate may be indicative of the level of the PFAS compound in the sample.

Provided herein is a method of detecting a PFAS compound in a sample. The method may comprise contacting the sample with the composition or the PFAS detection system. The PFAS binding protein may be bound to an electroactive surface to create a PFAS binding protein-bound base. The contacting may be under conditions that allow the PFAS binding protein to bind the PFAS compound to result in a PFAS binding protein-PFAS compound conjugate. The method may comprise measuring electrochemical signals with a transducer before and after binding of the PFAS compound to the PFAS binding protein-bound base, and calculating a difference between the electrochemical signals. A difference between the electrochemical signals before and after binding of the PFAS compound to the PFAS binding protein-bound base may correlate to the amount of the PFAS compound in the sample.

Provided herein is a method of detecting a PFAS compound in a sample. The method may comprise contacting the sample with the composition under conditions suitable for the PFAS compound to bind to the PFAS binding protein to generate a PFAS compound-PFAS binding protein conjugate. The PFAS binding protein may be bound to a surface of an optical planar waveguide. The method may comprise exposing the waveguide comprising the PFAS compound-PFAS binding protein conjugate to a sensing beam of polarized light. The method may comprise optically combining the sensing beam with an adjacent reference beam of polarized light to generate an interference pattern. The interference pattern may be indicative of a change in a speed of the sensing beam. The degree of change in the speed of the sensing beam may be indicative of the amount of the PFAS compound in the sample.

In the methods, the PFAS binding protein may be the anti-PFAS antibody or a biosimilar thereof. The anti-PFAS antibody may comprises a heavy chain variable region comprising the sequence set forth in SEQ ID NO: 17 and a light chain variable region comprising the sequence set forth in SEQ ID NO: 22. The anti-PFAS antibody may comprise a heavy chain variable region comprising the sequence set forth in SEQ ID NO: 19 and a light chain variable region comprising the sequence set forth in SEQ ID NO: 24. The detecting may be by an enzyme-linked immunosorbent assay (ELISA) or an electrochemical method.

In the methods, the limit of detection of the method may be no greater than a single digit parts per trillion. The sample may be a water sample. The water sample may be pre-treated, which may be accomplished by concentrating the PFAS compound before the contacting step. The pre-treatment may comprise removing inhibitors of the binding of the PFAS binding protein and the PFAS compound.

In the methods, the PFAS compound in the sample may comprise one or more straight chain perfluorinated carboxylic acid-or sulfonic acid molecules comprising a chain of 4 to 10 carbons in length. The PFAS in the sample may comprise one or more of perfluorobutanoic acid (PFBA), perfluoropentanoic acid (PFPeA), perfluorohexanoic acid (PFHxA), perfluoroheptanoic acid (PFHpA), perfluorooctanoic acid (PFOA), perfluorononanoic acid (PFNA), perfluorodecanoic acid (PFDA), perfluorobutane sulfonate (PFBS), perfluoropentane sulfonic acid (PFPeS), perfluorohexanesulphonic acid (PFHxS), perfluoroheptanesulfonic acid (PfHpS), perfluorooctane sulfonic acid (PFOS), perfluorononanesulfonic acid (PFNS), and perfluorodecane sulfonic acid (PFDS). The PFAS compound in the sample may comprise one or more of PFBA, PFPeA, PFHxA, PFHpA, PFOA, PFNA, and PFDA. The PFAS in the sample may comprise one or more of PFOA, PFOS, PFHxS, and PFBA. The PFAS in the sample may comprise PFOA.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a cartoon showing the competitive ELISA assay used with anti-PFOA polyclonal antibodies in this work.

FIG. 1B is a cartoon showing the development of a competitive ELISA assay.

FIG. 2A is a graph showing at least one antibody from a polyclonal mix bound to the PFOA-BSA conjugate.

FIG. 2B is a graph showing the sensitivity of polyclonal antibodies to PFOA versus octanoic acid.

FIG. 2C shows the results of competitive ELISA of polyclonal antibodies with PFOA-BSA versus free PFOA.

FIG. 2D shows the results of testing the polyclonal antibodies with PFOA-BSA and free PFOA.

FIG. 3 shows the results of indirect ELISA for five isolated clones for binding affinity to BSA-PFOA, wherein mAB3 and mAB5 were identified as candidates for ultrasensitive and specific binding to PFAS molecules.

FIG. 4A displays competitive ELISA results showing dose response of mAB3 and mAB5 to biotin-PFOA.

FIG. 4B displays competitive ELISA results showing dose response of mAB3 and mAB5 to HRP-PFOA.

FIG. 5 shows competitive ELISA results showing specificity of mAB3 and mAB5 binding to PFOA as compared to octanoic acid (OA).

FIG. 6 is a flow chart outlining three methods for testing anti-PFOA antibody interaction with PFOA/PFOA in water samples.

FIG. 7 is a flow chart outlining a field sensor for detecting single ppt levels of PFAS compounds.

FIG. 8 shows competitive ELISA results comparing PFOA binding affinity of mAB3 isolated from serum versus mAB3 purified using protein A and gel filtration chromatography and filtered through a 0.22 μm filter.

FIG. 9 shows that mAB3 can bind to PFOS and other PFAS compounds in addition to PFOA.

FIG. 10 shows an exemplary schematic representation of Surface Plasmon Resonance (SPR) using an anti-PFAS antibody. It is understood that the anti-PFOA/anti-PFAS antibody need not be limited to the specific embodiments involving mAB3 or mAB5.

FIG. 11 shows the Surface Plasmon Resonance (SPR) interaction between mAB3 and PFOA.

FIG. 12 shows the Surface Plasmon Resonance (SPR) interaction between mAB5 and PFOA.

FIG. 13 shows the principle of interferometry used in a biosensor.

FIGS. 14A-B show interferometry binding of mAB5 to PFOA. FIG. 14A shows the signal of mAB5 when exposed to buffer without PFOA (negative control) and FIG. 14B shows the signal of mAB5 when exposed to 2 ppb PFOA.

FIG. 15 shows interferometry binding of mAB5 to groundwater containing approximately 7.5 ppb of PFOA.

### DETAILED DESCRIPTION

Disclosed herein are PFAS binding proteins, including antibodies, and their use in environmental bioremediation. The focal activity of interest for this technology is the ability to use the antibody for detecting perfluorooctanoic acid (PFOA) and other PFAS compounds. The inventors have discovered PFAS binding proteins, particularly anti-PFOA antibodies, with an unexpected binding affinity to PFOS. Antibodies were developed in New Zealand white rabbits (Oryctolagus cuniculus) by immunizing the rabbits with PFOA tethered to carrier protein at the carboxylic acid end. Since protein tethering protected the carboxylic acid group on PFOA, it was hypothesized that, first, PFAS binding proteins will only recognize the fluorinated carbon chain of PFOA and, second, the binding proteins will also recognize PFOS, because its fluorinated carbon chain is identical to PFOA. Results indicated that developed antibodies had very similar affinity to both PFOA and PFOS. Developed antibodies were also tested for their specificity of binding to PFAS compounds by comparing this binding affinity to analogs like octanoic acid (OA). The results clearly indicate that the polyclonal antibodies developed by this approach have higher binding affinity to PFOA and PFOS than OA. Further, competitive ELISA methods were designed using colorimetric outputs to enable quantification, and to determine the dynamic range including the limit of detection of PFOA antibodies. Monoclonal antibodies were used for these purposes. BSA-PFOA was used as the substrate. Another substrate that was tested was PFOA-biotin. Also, these monoclonal antibodies were used to determine their individual sensitivity to OA. This was done by determining the range of OA binding and the overlap with PFOA affinity.

In this disclosure, the production of antibodies that bind specifically to PFAS molecules (5-78 times lower binding affinity to octanoic acid (OA)) is described. The Limit of Detection (LoD) of two of the antibody candidates in colorimetric assays is about 500 ppt when tested in antibodies isolated from serum. The LoD is about 1 ppt when the antibodies are produced in vitro and purified using Protein A and gel filtration chromatography. This is the most sensitive demonstration of a bio-inspired detection system of a PFAS compound.

### 1. Definitions

Before the present compositions and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

The terminology used herein is for the purpose of describing particular embodiments only and is not intended to be limiting. As used in the specification and the appended claims, the singular forms “a,” “an” and “the” include plural referents unless the context clearly dictates otherwise. Thus, for example, reference to a kit containing “a monoclonal antibody” includes a mixture of two or more monoclonal antibodies, reference to “an antibody” includes reference to two or more of such antibodies, and reference to “a PFAS” includes reference to a mixture of two or more PFAS.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

As used herein, the term “antibody” is intended to denote an immunoglobulin molecule derived from any mammal that possesses a “variable region” antigen recognition site. The term “variable region” is intended to distinguish such domain of the immunoglobulin from domains that are broadly shared by antibodies (such as an antibody Fc domain). The variable region comprises a “hypervariable region” whose residues are responsible for antigen binding. The hypervariable region comprises amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelid antibodies, single chain antibodies, disulfide-linked Fvs (scFv), intrabodies, and anti-idiotypic (anti-Id) antibodies (including, e.g., anti-Id and anti-anti-Id antibodies to antibodies disclosed herein). In particular, such antibodies include immunoglobulin molecules of any type (e.g., IgG, IgE, IgM, IgD, IgA and IgY), class (e.g., IgG1, IgG2, IgG3, IgG4, IgA1 and IgA2), or subclass of the foregoing.

An antibody may also be produced in an animal from the camelid family, which includes llamas. Such animals produce a subclass of IgGs that possess a single heavy-chain variable domain. This heavy-chain variable domain has demonstrated the ability to function as an independent antigen-binding domain with similar affinity as a conventional IgG. These heavy chain variable domains can be expressed as a single domain, known as a variable heavy domain of heavy chain antibody (VHH) or nanobody, with a molecular weight 10% of the full IgG. The terms “VHH domain” and “nanobody,” are used herein interchangeably. The terms are used in their broadest sense, and not limited to a specific biological source or to a specific method of preparation. Nanobodies may display superior solubility, solution stability, temperature stability, and strong penetration into tissues, may be easily manipulated with recombinant molecular biology methods, and may possess robust environmental resilience to conditions detrimental to conventional IgG antibodies. In addition, nanobodies may be weakly immunogenic which reduces the likelihood of adverse effects compared to other single domain antibodies such as those derived from sharks or synthetic platforms.

As used herein, the term “antigen binding fragment” of an antibody refers to one or more portions of an antibody that contain the antibody's CDR and optionally the framework residues that comprise the antibody's “variable region” antigen recognition site and exhibit an ability to immunospecifically bind antigen. Such fragments include Fab′, F(ab′)2, Fv, single chain (ScFv), and mutants thereof, naturally occurring variants, and fusion proteins comprising the antibody's “variable region” antigen recognition site and a heterologous protein (e.g., a toxin, an antigen recognition site for a different antigen, an enzyme, a receptor or receptor ligand, etc.). As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

As used herein, “comprising,” “including,” “containing,” “characterized by,” and grammatical equivalents thereof are inclusive or open-ended terms that do not exclude additional, unrecited elements or method steps.

As used herein, “PFAS” means per-or polyfluoroalkyl compounds, “PFOA” means perfluorooctanoic acid; “PFOS” means perfluorooctanesulfonic acid, “PFHxS” means perfluorohexanesulfonic acid, “PFBA” means perfluorobutanoic acid, “PFPeA” means perfluoropentanoic Acid, “PFHxA” means perfluorohexanoic acid, “PFHpA” means perfluoroheptanoic acid, “PFNA” means, perfluorononanoic acid, “PFDA” means perfluorodecanoic acid, and “OA” means octanoic acid. As used herein, a “biosimilar” is an antibody that is highly similar to a reference antibody in both molecular structure and bioactivity, but bioactivity may differ from the reference antibody in some way that does not substantially affect bioactivity.

### 2. PFAS Binding Proteins

Provided herein is a PFAS binding protein. The PFAS binding protein may bind to one or more PFAS compounds. The PFAS compounds may comprise a straight chain PFAS compound. The PFAS compounds may comprise one or more straight chain perfluorinated carboxylic- or sulfonic acid molecules of 4 to 10 carbons in length. The PFAS compounds may comprise one or more of perfluorobutanoic acid (PFBA), perfluoropentanoic acid (PFPeA), perfluorohexanoic acid (PFHxA), perfluoroheptanoic acid (PFHpA), perfluorooctanoic acid (PFOA), perfluorononanoic acid (PFNA), and perfluorodecanoic acid (PFDA). The PFAS compounds may also comprise one or more of perfluorobutane sulfonate (PFBS), perfluoropentane sulfonic acid (PFPeS), perfluorohexanesulphonic acid (PFHxS), perfluoroheptanesulfonic acid (PfHpS), perfluorooctane sulfonic acid (PFOS), perfluorononanesulfonic acid (PFNS), and perfluorodecane sulfonic acid (PFDS). In one example, the PFAS compounds comprise one or more of PFOA, PFOS, PFHxS, and PFBA. In another example, the PFAS compounds comprise PFOA.

The binding protein may be an antibody or a monobody. The antibody may be a polyclonal antibody or a monoclonal antibody, a biosimilar thereof, or an antigen binding fragment thereof. The antibody may be a heterotetrameric antibody or a single domain antibody. The single domain antibody may be a VHH or nanobody, or a single chain variable fragment [scFv]). The antibody may be produced in a chicken, mouse, rat, rabbit, guinea pig, sheep, pig, goat, horse, or camelid. The camelid may be a camel, llama, or alpaca. The biosimilar of an antibody may be configured for binding to a PFOA or PFAS and may have a molecular structure substantially similar to that of a reference antibody. The biosimilar may comprise one or more truncations, deletion variants, or substitutions relative to the reference antibody, or may include additional amino acid residues attached thereto.

The heavy chain of the antibody may comprise a heavy chain constant region, and the light chain of the antibody may comprise a light chain constant region. Constant regions are known in the art. The antibody may be a rabbit antibody, and may be an IgA, IgD, IgE, or IgM isotype. In one example, the antibody is a rabbit IgG antibody. The light chain of the rabbit antibody may comprise an IgG-K constant region, which may be a K1 or K2 isotype, or may comprise an IgG-λ constant region. The IgG-κ K1 light chain may be a b4, b5, b6, or b9 allotype. In another example, the antibody is a mouse antibody, which may be an IgA, IgD, IgE, IgG, or IgM isotype. In a further example, the antibody may be humanized and may be an IgG, IgE, IgM, IgD, IgA and IgY), class (e.g., IgG1, IgG2, IgG3, IgG4, IgA1 and IgA2 isotype. The heavy chain of an antibody disclosed herein may comprise one of the foregoing Ig constant regions.

In one example, the antibody is a rabbit antibody, which may be a monoclonal antibody. The antibody may comprise a heavy chain encoded by a nucleotide sequence comprising the sequence set forth in one of SEQ ID NOs: 1, 3, 5, 7, and 9. The heavy chain may comprise a heavy chain variable region comprising one of the following amino acid sequences:

(SEQ ID NO: 15)
QSVKESEGGLFKPTDTLTLTCTASGFSLSSYGVSWVRQAPGNGLEWIGSI
EDGGSAYYASWAKSRSTITSNTNLNTVTLQMTSLTAADTATYFCGSLSIG
SAYPGIWGPGTLVTVSS
(SEQ ID NO: 16)
QSVKESEGGLFKPTDNLTLTCAVSRFSLSSYGVSWVRQAPGNGLEWIGYI
YTDGSAYYASWAKSRSTITRNTNLNTVTLKMTSLTAADTATYFCARHSSG
WGIDYFNVWGPGTLVTVSS
(SEQ ID NO: 17)
QSLEESEGGLFKPGGTLTLTCTVSGFSLNIYSISWVRQAPGKELEWIGTM
SNVDATYYANWAKSRSTITRNINESTVTLKMPSLTVADTATYFCARYIYG
AGYDIWGPGTLVTVSS
(SEQ ID NO: 18)
QEQLEESRGGLIKPTDTLTLTCTASGFSLSNYGINWVRQAPGEGLEWIGA
IRGSGFIYYATWAKSRSTITRNTNLNTVTLKMTSLTAADTATYFCARRDG
LYSAMDPWGPGTLVTVSS
(SEQ ID NO: 19)
QSVKESEGGLFKPTDTLTLTCTASGFSLSSYGVIWVRQAPGKGLQWIGFI
NIYGTPSYASWAMGRSTITGNTNLNTVILKMTSLTVADTATYFCASSVGS
DGYLYSSDIWGPGTLVTVSS

The antibody may comprise a light chain encoded by a nucleotide sequence comprising the sequence set forth in one of SEQ ID NOs: 2, 4, 6, 8, and 10. The light chain may comprise a light chain variable region comprising one of the following amino acid sequences:

(SEQ ID NO: 20)
AIVMTQTPSSKSVPVGDTVTIKCQASQSIGTRLAWFQQKPGQPPKRLIYS
ASTLASGVPSRFSGSGSGTQFTLTISDVVCDDAAAYYCGGYKSSSSDGIA
FGGGTEVVVKG
(SEQ ID NO: 21)
AAVLTQTPSSVSAAVGGTVTINCQASQSVYNNNYLSWYQQKPGQPPKLLI
YLASTLASGVPSRFSGSGSGTEFTLTISDLECDDGATYYCAGGVTSSDRP
FGGGTEVVVKG
(SEQ ID NO: 22)
ADIVMTQTPASVEAAVGGTVTIKCQASQSISSWLSWYQQKPGQPPKLLIY
KASTLASGVPSRFKGSGSGTEYTLTISGVQCADAATYYCQSVYYSSSTNY
GNTFGGGTEVVVKG
(SEQ ID NO: 23)
DPVLTQTPSSTSAAVGGTVTISCQSSDSVYKNNYLAWFQQKPGQPPKVLI
YHASKLASGVPSRFSGSGSGTQFTLTISGVQCDDAATYYCLGEYSDTHVE
GGGTEVVVKG
(SEQ ID NO: 24)
DPVLTQTPSSVSAAVGGTVTINCQASQSVYNNKCLAWYQQKPGQPPKLLI
YGASTLASGVPSRFSGSGSGTQFTLTISDLECDDAATYYCAGGYYSEIHF
GGGTEVVVKG

In one example, the heavy chain comprises the sequence set forth in SEQ ID NO: 15 and the light chain comprises the sequence set forth in SEQ ID NO: 20. The antibody may be mAB1. In one example, the heavy chain comprises the sequence set forth in SEQ ID NO: 16 and the light chain comprises the sequence set forth in SEQ ID NO: 21. The antibody may be mAB2. In one example, the heavy chain comprises the sequence set forth in SEQ ID NO: 17 and the light chain comprises the sequence set forth in SEQ ID NO: 22. The antibody may be mAB3. In one example, the heavy chain comprises the sequence set forth in SEQ ID NO: 18 and the light chain comprises the sequence set forth in SEQ ID NO: 23. The antibody may be mAB4. In one example, the heavy chain comprises the sequence set forth in SEQ ID NO: 19 and the light chain comprises the sequence set forth in SEQ ID NO: 24. The antibody may be mAB5.

In one example, the heavy chain comprises the heavy chain variable region of SEQ ID NO: 11 or 13. In a further example, the light chain comprises the light chain variable region of SEQ ID NO: 12 or 14. In another example, the heavy chain comprises the heavy chain variable region of SEQ ID NO: 11 and the light chain comprises the light chain variable region of SEQ ID NO: 12, and the antibody may be mAB3. In a further example, the heavy chain comprises the heavy chain variable region of SEQ ID NO: 13 and the light chain comprises the light chain variable region of SEQ ID NO: 14, and the antibody may be mAB5. The heavy and light chains of the antibody may lack signal peptides.

The biosimilar to the anti-PFAS antibody may comprise amino acid changes as compared to a reference anti-PFAS antibody while maintaining the desirable antigen-binding characteristics. For example, certain amino acid residues may be substituted for other amino acid residues in a protein structure without appreciable loss of interactive binding capacity with structures such as, for example, antigen-binding regions of antibodies. Since it is the interactive capacity and nature of a protein that defines that protein's biological functional activity, certain amino acid sequence substitutions can be made in an amino acid sequence and nevertheless obtain a protein with like properties. It is thus contemplated that various changes may be made in the sequence of a biosimilar antibody without appreciable loss of its biological utility or activity.

It is also well understood by the skilled artisan that inherent in the definition of a biologically functional equivalent protein or peptide is the concept that there is a limit to the number of changes that may be made within a defined portion of the molecule and still result in a molecule with an acceptable level of equivalent biological activity. It is also well understood that where certain residues are shown to be particularly important to the biological or structural properties of a protein or peptide, e.g. residues in active sites, such residues may not generally be exchanged.

Amino acid substitutions are generally based on the relative similarity of the amino acid side-chains relative to, for example, their hydrophobicity, hydrophilicity, charge, size, and the like. An analysis of the size, shape, and type of the amino acid side-chains reveals, for example, that arginine, lysine, and histidine are all positively charged residues; that alanine, glycine, and serine are all of similar size; and that phenylalanine, tryptophan, and tyrosine all have a generally similar shape. Therefore, based upon these considerations, the following conservative substitution groups or biologically functional equivalents have been defined: (a) Cys; (b) Phe, Trp, Tyr; (c) Gln, Glu, Asn, Asp; (d) His, Lys, Arg; (e) Ala, Gly, Pro, Ser, Thr; and (f) Met, Ile, Leu, Val. M. Dayhoff et al., Atlas of Protein Sequence and Structure (Nat'l Biomed. Res. Found., Washington, D.C., 1978), hereby incorporated by reference.

To effect more quantitative changes, the hydropathic index of amino acids may be considered. Each amino acid has been assigned a hydropathic index on the basis of its hydrophobicity and charge characteristics, which are as follows: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art. J. Kyte & R. Doolittle, A simple method for displaying the hydropathic character of a protein, 157 J. Mol. Biol. 105-132 (1982), incorporated herein by reference. It is known that certain amino acids may be substituted for other amino acids having a similar hydropathic index or score and still retain a similar biological activity. In making changes based on the hydropathic index, amino acids whose hydropathic indices are within +2, +1 or +0.5 of a reference protein may be substituted.

It is also understood that an amino acid can be substituted for another having a similar hydrophilicity value and still obtain a biologically equivalent protein. As detailed in U.S. Pat. No. 4,554,101, the following hydrophilicity values have been assigned to amino acid residues: arginine (+3.0); lysine (+3.0); aspartate (+3.0 +1); glutamate (+3.0 +1); serine (+0.3); asparagine (+0.2); glutamine (+0.2); glycine (0); threonine (−0.4); proline (−0.5 +1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, amino acids whose hydrophilicity values are within +2, +1 or +0.5 of a reference protein may be substituted.

### 3. PFAS Detection Systems and Methods of Detecting PFAS

Provided herein is a PFAS detection system comprising one or more PFAS binding proteins disclosed herein. In one example, the system comprises the PFAS antibody, biosimilar thereof, or antigen binding fragment thereof. The detection system may comprise a solution comprising the PFAS binding protein or a solid substrate comprising the PFAS binding protein. The PFAS binding protein may be attached to the solid substrate, and the attachment may be covalent or non-covalent. The solid substrate may comprise an electroactive surface. A cartridge may comprise the solid substrate. The solid substrate may comprise a coating of protein A, protein G, protein L, or a combination of protein A and protein G, and the anti-PFAS antibody may be bound to the coating. The solid substrate may comprise a coating of streptavidin, NeutrAvidin, or a biotin-binding protein, and the anti-PFAS antibody may be biotinylated.

The detection system may comprise a solid substrate comprising a secondary antibody. The secondary antibody may be attached to the solid substrate. The solid substrate may be a cartridge. The secondary antibody may bind to the PFAS binding molecule. In one example, the secondary antibody is an anti-IgG antibody.

Components of the detection system may be provided as a kit. The kit may comprise one or more of the PFAS binding molecule in solution, the solid substrate comprising the anti-PFAS antibody, the solid substrate comprising the secondary antibody, a conjugated analyte, an enzymatic detection molecule, an enzyme substrate, and one or more negative or positive controls. The conjugated analyte may be a biotin-conjugate analyte.

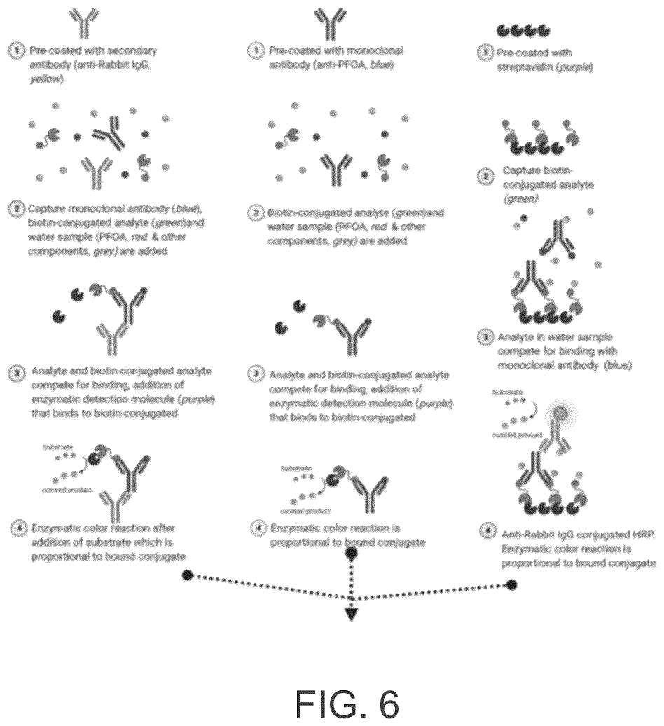

PFAS binding proteins disclosed herein may be deployed in a biosensor system to detect low concentrations of PFAS compounds. Examples of four potential methods that deploy the antibodies are set out in FIG. 6.

In a first example, provided herein is a device and system comprising the PFAS binding protein. The device, which may comprise a cartridge, may comprise a surface coated with a secondary antibody, which may be an anti-rabbit IgG. The system may comprise a PFAS binding protein, which may be the anti-PFAS monoclonal antibody, and a biotin-conjugated analyte. A sample suspected of containing PFAS and other known and/or unknown components may be introduced to the cartridge. The PFAS and biotin-conjugated analyte may compete for binding, and an enzymatic detection molecule that binds the biotin-conjugated analyte may be added. An enzyme substrate may be added, and an enzymatic color reaction may proceed. The amount of color may be proportional to the amount of bound conjugate.

In a second example, the device, which may comprise a cartridge, may comprise a surface coated with the PFAS binding protein, which may be an anti-PFOA or anti-PFAS monoclonal antibody. A biotin-conjugated analyte and a sample suspected of containing PFOA or PFAS and other known and/or unknown components may be added to the cartridge. The PFOA or PFAS and biotin-conjugated analyte may compete for binding, and an enzymatic detection molecule that binds the biotin-conjugated analyte may be added. An enzymatic color reaction may occur, and the amount of color may be proportional to the amount of bound conjugate.

In a third example, the device, which may comprise a cartridge, may comprise a surface coated with streptavidin. A biotin-conjugated analyte may be introduced to the cartridge and captured by biotin-streptavidin binding. The anti-PFAS antibody and a water sample suspected of containing PFOA or PFAS and other known and/or unknown components may be introduced to the cartridge, and the PFOA or PFAS in the water may compete for binding with the anti-PFAS antibody. An anti-rabbit IgG conjugated with HRP may be added, and an enzymatic color reaction may occur. The amount of color may be proportional to the amount of bound conjugate.

In a fourth example, the device may comprise the anti-PFAs antibody bound to an electroactive surface, which may create a baseline electrochemical property. A water sample suspected of containing PFAS may be passed over the surface, where PFAS may preferentially bind to the antibodies. The binding may alter the electrochemical properties of the surface. A voltametric reading using a potentiostat may deliver an altered signal, which may be measured by a transducer and displayed on a signal display readout. The amount of PFAS binding events may be proportional to the altered electrochemical signal measured. The anti-PFAS antibody may comprise an electroactive label conjugate. The electroactive label may be a ferrocene derivative. The electroactive label may be ferrocenecarboxylic acid, anthraquion-one 2-carboxylic acid, thionine, tris(2,2′-bipyridine-4,4′-dicarboxylic acid)cobalt(III), tris(bipyridine)ruthenium(II) with an N-succinimidyl ester group, or an iron heme group in horseradish peroxidase. The electroactive label may be phenazine dye, neutral red, toluidine blue, Prussian blue, methylene blue, azure A, thionine, anthraquinone, or tris(bipyridine)ruthenium(II) [Ru(bpy)₃]²⁺ The anti-PFAS antibody may be covalently bound to the electroactive surface. In one example, the electroactive surface comprises a gold surface. The gold surface made be modified with 3,3′-dithiobis (sulfosuccinimidyl) propionate (DTSSP). In another example, the anti-PFAS antibody is immobilized and is thiolated. In a further example, the anti-PFAS antibody is immobilized on a protein G layer on the electroactive surface.

In a fifth example, the device may combine two very sensitive methods, waveguiding and interferometry, to form waveguide interferometry technology for use in rapid low-level detection sensing applications. As the basis of a PFAS sensor, a PFAS binding protein, which may be an anti-PFAS antibody, may be coupled to the waveguide surface. As PFAS is introduced to the functionalized waveguide, the binding of PFAS to the antibody may displace a sample solution near the waveguide surface, changing the light beam's velocity; an adjacent reference beam may be left unperturbed and optically combined with the sensing beam to measure the velocity change. This may be create an interference pattern that shifts as the refractive index changes, producing a corresponding change in the relative phase measurement.

Sensitivity and stability studies of the various components of the device may be studied to select the one or more methods that provide optimal results.

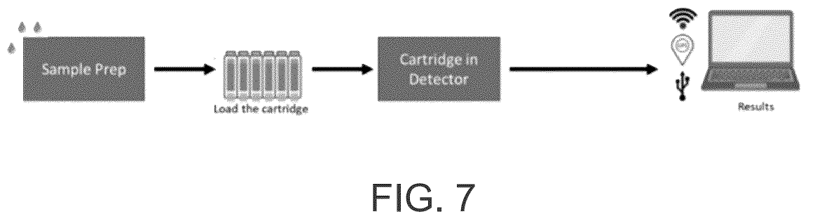

In a further example, FIG. 7 shows a flow chart outlining a potential field sensor for detecting single ppt levels of PFAS compounds in a water sample. A water sample may be obtained and loaded into the cartridge, where the enzymatic color reaction is developed. The cartridge may be loaded into a detector, which may detect the level of color reaction, which may be proportional to the amount of analyte in the water sample. The output of the detector may be loaded into a computing device, which may analyze the results.

Also provided herein is a method of detecting PFAS. The method may comprise pre-treating a water sample to concentrate the PFAS compounds and to degrade potential inhibitors. This approach may detect at least 4 ppt, and, preferably, single digit ppt of PFAS compounds, and may increase specificity of the detection method towards PFAS compounds. The stability of the antibodies and their response to inhibitory components in a “real world” matrix may be tested. After addressing these variables, various scenarios of field kit development may be tested. One such design may comprise the anti-PFAS antibody immobilized directly on electrodes (which may be termed an immunosensor). The electrodes may be contained in a flow cartridge. A liquid sample, such as groundwater, suspected of containing PFAS may be added to facilitate washing, incubation, and measurement steps. A field kit comprising the immunosensor may be weather resistant; may be battery powered; may have GPS capabilities; or may have capacity to measure four samples simultaneously; or a combination of the foregoing. Upon completion of the incubation and washing, a software program may measure the amount of PFAS in the sample and produce results that can be visualized or exported in a spreadsheet format.

The resulting field kit may be used for detection of PFAS compounds at a contaminated site. The field kit may be used to draw PFAS plume maps without having to spend valuable resources and time in sending samples to a laboratory for sensitive testing. A method of detecting PFAS disclosed herein, if combined with a field kit, may reduce the number of samples that have to be sent out for expensive laboratory testing and reduce the amount of time it takes to map contaminated sites.

A device disclosed herein may be used to detect PFAS using surface plasmon resonance. The device may comprise a surface, which may comprise a metal. The surface may be functionalized to bind proteins such as antibodies. A PFAS binding protein disclosed herein may be bound to the surface. In another example, a device disclosed herein may be used to detect PFAS using waveguide interferometry. The device may comprise a waveguide, which may be functionalized to bind proteins such as antibodies. A PFAS binding protein disclosed herein may be bound to the waveguide.

The present invention has multiple aspects, illustrated by the following non-limiting examples.

### Example 1

### Anti-PFAS Antibodies

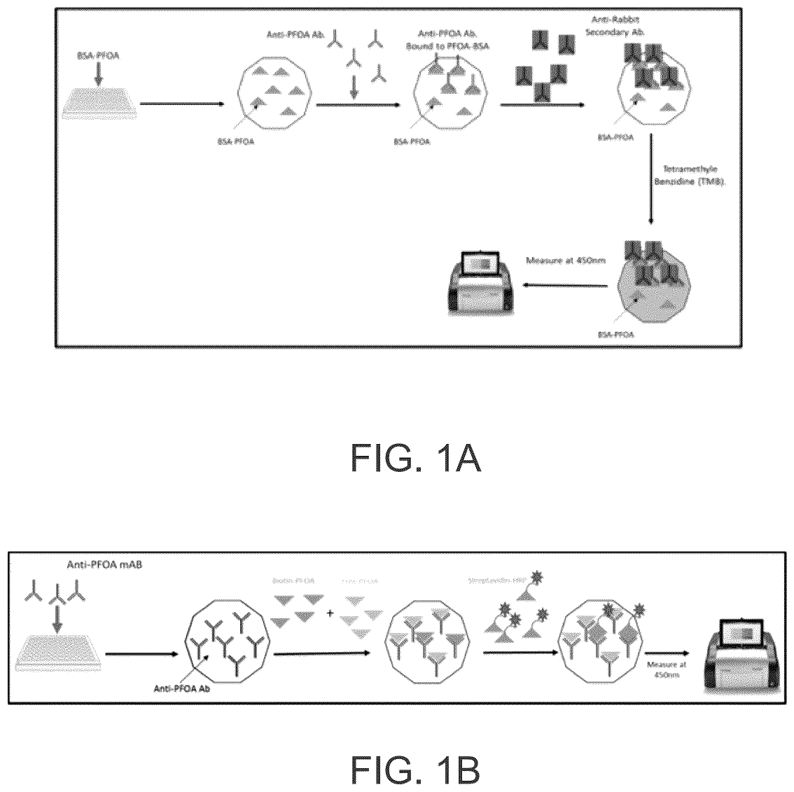

Polyclonal rabbit antibodies to PFOA were prepared according to methods well known in the art. E. Harlow & D. Lane, Antibodies: A Laboratory Manual (1988) (published by Cold Spring Harbor Press, Cold Spring Harbor, N.Y.). These polyclonal antibodies were tested for their binding affinity to PFOA (FIG. 1A). PFOA was conjugated to bovine serum albumin by methods well known in the art. G.T. Hermanson, Bioconjugate Techniques (Academic Press, 785 pp., 1995). S.S. Wong, Chemistry of Protein Conjugation and Cross-Linking (CRC Press, 340 pp., 1991); G.T. Hermanson et al., Immobilized Affinity Ligand Techniques (Academic Press, 450 pp., 1992). Microplates were treated with BSA-PFOA conjugate and incubated. After washing with buffer, blocking solution was added followed by varying dilutions of monoclonal antibodies (mABs). The plate was then further incubated. After washing again, anti-rabbit secondary antibodies were added. A color reaction was developed using tetramethyl benzidine (TMB), and the reaction was read at 450 nm.

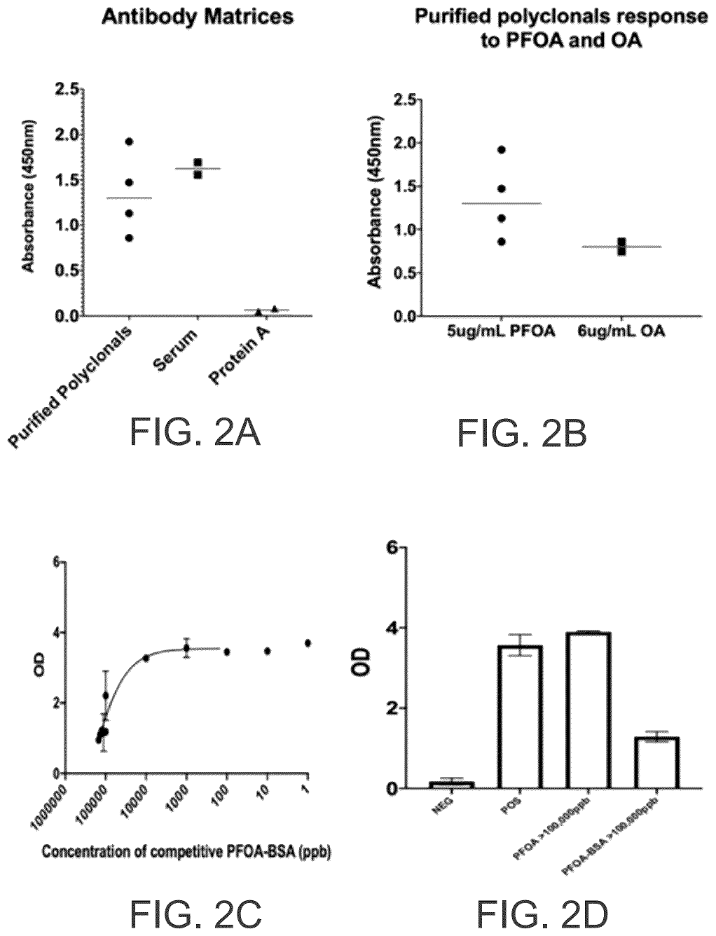

FIG. 2A shows that at least one antibody from a polyclonal antibody mixture bound to the PFOA-BSA conjugate. Similar absorbance was seen between serum and the polyclonal antibody mixture, but a reduced signal was reported for protein A purified antibodies.

FIG. 2B shows the sensitivity of the polyclonal antibodies to PFOA versus octanoic acid (OA). The polyclonal antibodies showed a higher binding affinity to PFOA than OA. Even at higher concentrations the signal response of OA-antibody binding was lower than the affinity of PFOA to the antibodies.

FIG. 2C shows the results of competitive ELISA of the polyclonal antibodies with PFOA-BSA versus free PFOA. FIG. 2D shows the results of testing the polyclonal antibodies with PFOA-BSA and free PFOA.

Anti-PFOA monoclonal antibodies were also prepared with New Zealand White Rabbit cells according to methods well known in the art. G. Kohler & C. Milstein, Continuous cultures of fused cells secreting antibody of pre-defined specificity, 256 Nature 495-97 (1975); Wunderlich et al., 17 Eur. J. Cancer Clin. Oncol. 719 (1981); Schlom et al., 77 Proc. Nat'l Acad. Sci. USA 6841 (1980) (human monoclonal antibodies).

Initial screening of the anti-PFOA monoclonal antibodies prepared in this way identified five distinct antibodies that detected PFOA. The variable regions of the antibodies were cloned and sequenced by nucleotide sequencing (both heavy and light chains) according to methods well known in the art. The antibody IDs and the corresponding sequences are listed in Table 1 and the Sequence Listing.

TABLE 1

| Antibody ID | Clone ID | Variable Chain | SEQ ID NO. |
|---|---|---|---|
| mAB1 | #29 | Heavy (HC) | 1 |
| | | Light (LC) | 2 |
| mAB2 | #18 | Heavy (HC) | 3 |
| | | Light (LC) | 4 |
| mAB3 | #24 | Heavy (HC) | 5 |
| | | Light (LC) | 6 |
| mAB4 | #20B | Heavy (HC) | 7 |
| | | Light (LC) | 8 |
| mAB5 | #2 | Heavy (HC) | 9 |
| | | Light (LC) | 10 |

FIG. 1B shows development of competitive ELISA using the monoclonal antibodies. Microplates were treated with anti-PFOA mABs. After incubation, the wells were exposed to a biotin-PFOA conjugate and free PFOA compounds, which compete for a finite number of antibody recognition sites. Next, streptavidin-HRP conjugate was added, which binds to the biotin-PFOA molecules. The resultant color reaction was measured spectrophotometrically. P. Langer et al., 78 Proc. Nat'l Acad. Sci USA 6633-37 (1981); A. Forster et al., 13 Nucleic Acids Res. 745-61 (1985); L. Riley et al., 5 DNA 333-37 (1986); M.D. Savage et al., Avidin-Biotin Chemistry: A Handbook (Pierce Chemical Co., 467 pp., 1992). The amount of signal was inversely proportional to the concentration of free-PFOA provided. This approach determined the dynamic range of antibody detection.

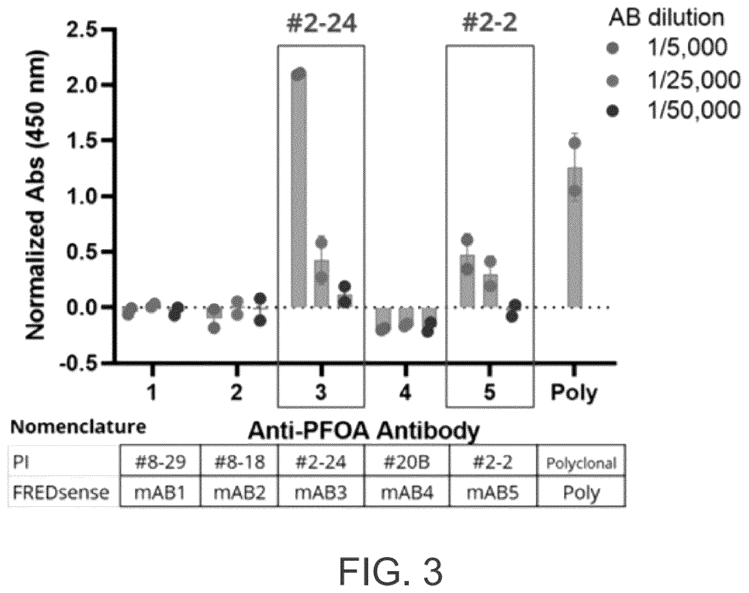

As shown in FIG. 3, initial indirect ELISA results indicated that mAB3 (Clone #24) and mAB5 (Clone #2) showed the best binding affinity to BSA-PFOA. The results were consistent over various antibody dilutions. The best binding affinity to BSA-PFOA for mAB3 was at dilutions of 1:5,000 and 1:25,000. The response at 1:25,000 was approximately four-fold lower. The response at 1:50,000 was very depressed. Antibody mAB5 showed a similar response to BSA-PFOA at 1:5,000 and 1:25,000 dilutions-the response was not proportional to concentration-while its response at 1:50,000 dilution was negligible. The three other antibodies (mAB1, mAB2, and mAB4) had no significant binding affinity to BSA-PFOA.

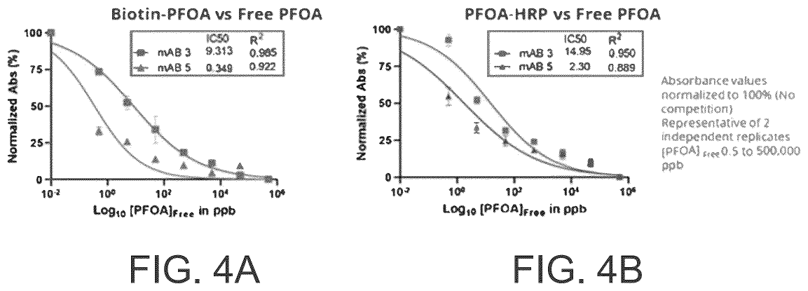

Antibodies mAB3 and mAB5 were then tested for dose response to PFOA using the competitive ELISA described in FIG. 1B. The assay was performed using two carrier molecules: (1) biotin (small carrier), and (2) horse radish peroxidase (HRP; large carrier). The results (FIGS. 4-1 and 4-2) indicated a better dynamic response to biotin-PFOA as compared to HRP-PFOA, as evidenced by the lower half-maximal inhibitory concentration (IC50) for mAB3 and mAB5 of 9.313 and 0.349, respectively. Also observed was the dynamic response of the assay to 0.5 ppb (500 ppt) of PFOA. This result indicates that the two antibodies can detect as low as 0.5 ppb of PFOA in the synthetic matrix under laboratory conditions.

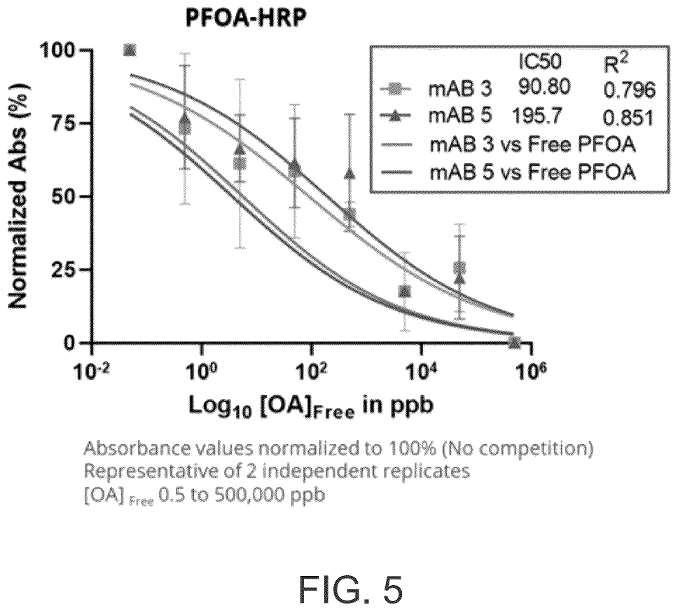

Antibodies mAB3 and mAB5 were also tested for specificity of binding to PFOA as compared to known analogs, such as OA. The results (FIG. 5) showed that both antibodies have less affinity to OA as compared to PFOA. While mAB3 showed six-fold lower affinity to OA as compared to PFOA, mAB5 displayed a 78-fold higher response to PFOA versus OA. This result demonstrates that these monoclonal antibodies are highly selective toward PFOA and show preferential binding affinity to PFOA at 0.5 ppb (500 ppt).

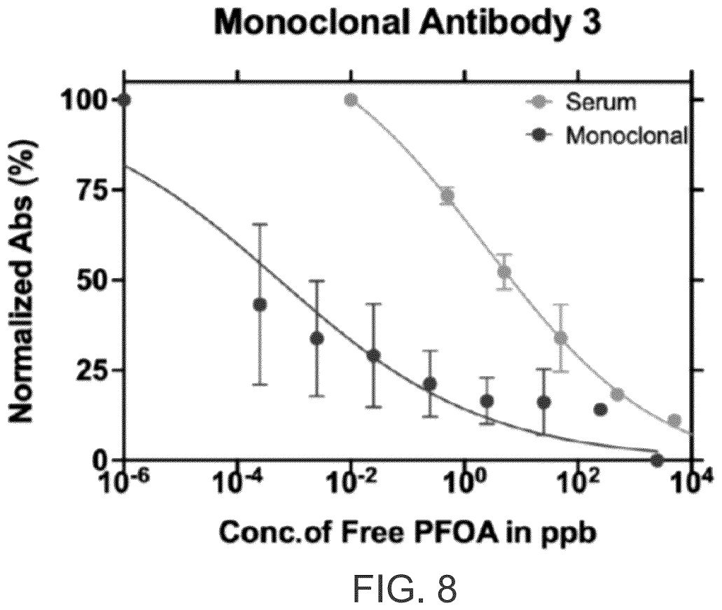

A plasmid containing the sequence for antibody mAB3 was expressed in Expi293 cells, and the resulting antibody was purified using Protein A and gel filtration chromatography, as is well known in the art. This antibody was tested for its binding affinity to PFOA using the competitive ELISA described in FIG. 1B. The results (FIG. 8) showed that the binding affinity of mAB3 in this purified form was about 1 ppt, as compared to a binding affinity of about 500 ppt with mAB3 isolated from rabbit serum.

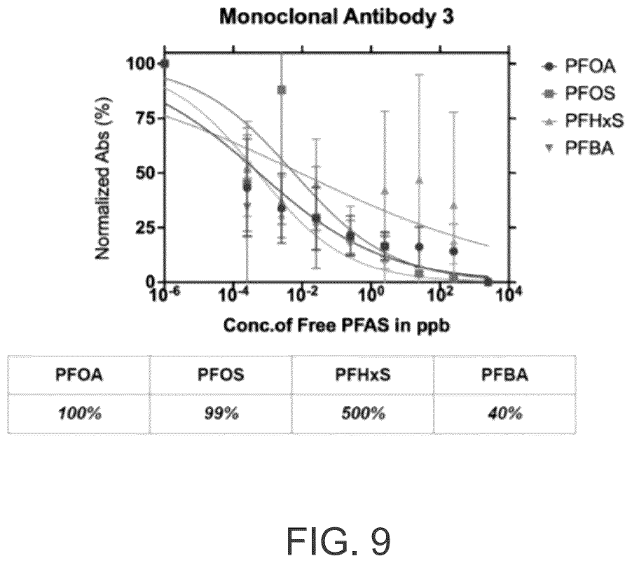

The mAB3 produced in vitro was also tested for its binding affinity to other PFAS compounds, including PFOS, PFHxS, and PFBA. The results (FIG. 9) indicated that the purified antibody, mAB3, has very good binding affinity to PFOS (99%) as compared to PFOA. This indicates that this antibody can be used to detect both PFOA and PFOS (total regulated PFAS) with this system.

### Example 2

### PFAS Binding Proteins Can Detect PFAS When Used in Surface Plasmon Resonance Detection Systems

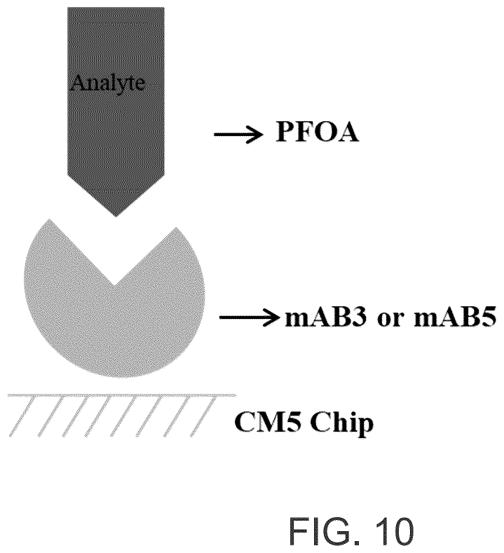

This example demonstrates that PFAS binding proteins disclosed herein successfully detect PFAS at low concentrations when implemented in a surface plasmon resonance (SPR) detection system. Surface plasmon resonance occurs when a directed light at a specific angle of incidence excites electrons in a thin metal sheet. As a result, the electrons travel parallel to the sheet. This is related to the refractive index of the material. Even a small change alters the response, allowing SPR to be used as a techniques for detection of physical interactions between analytes. FIG. 10 shows a schematic of an anti-PFAS antibody bound to a sensor chip to detect PFAS. In this example, the antibody is bound to a sensor chip that carries a matrix of carboxymethylated dextran covalently attached to a gold surface. In this example, the chip is a CM5 sensor chip.

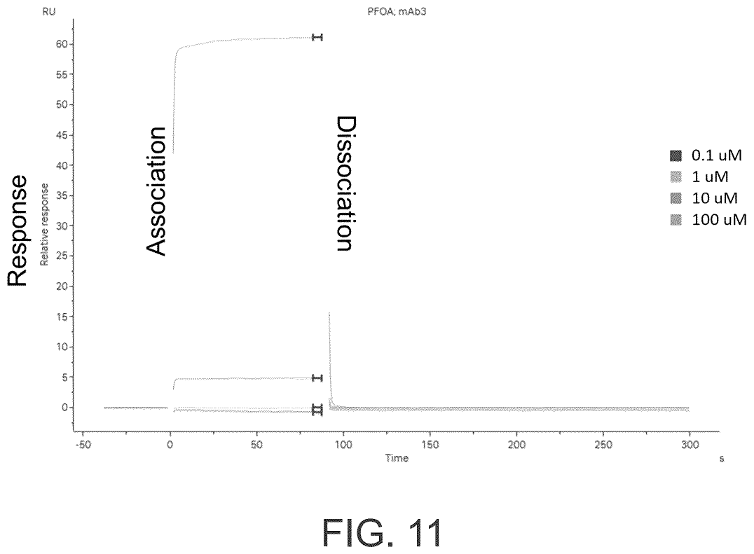

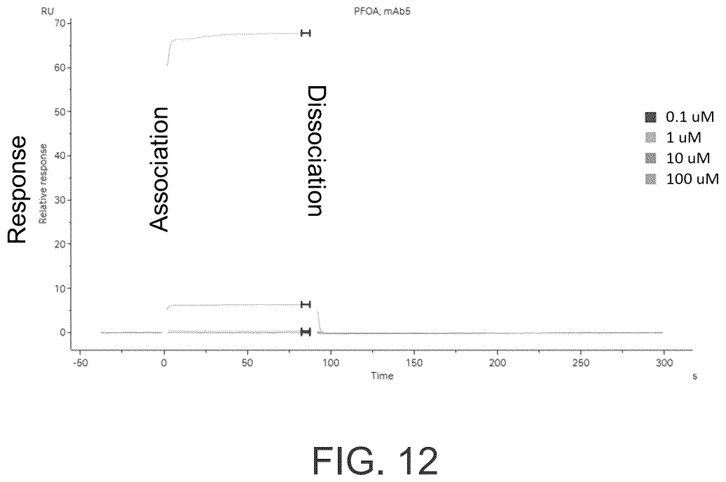

mAb3 (FIG. 11) and mAB5 (FIG. 12) were tested for binding with different concentrations of PFOA (100 μμM, 10 μM, 1 μM, 0.1 μM). The concentration of PFOA tested was between ˜41 ppm and 41 ppb. The results clearly demonstrate a concentration dependent signal between 41 ppm and 400 ppb for both antibodies. The binding was a steady-state interaction as observed by instant association and dissociation curve. This demonstrates that anti-PFAS binding proteins disclosed herein can be used to detect PFAS when implemented in SPR detection systems.

### Example 3

### PFAS Binding Proteins Can Detect PFAS When Used in Interferometry Detection Systems

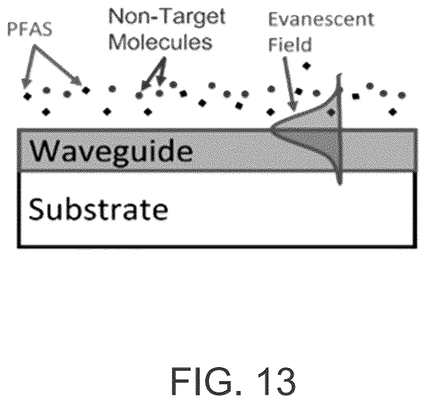

This example demonstrates that PFAS binding proteins disclosed herein successfully detect PFAS at low concentrations when implemented in an interferometry detection system. FIG. 13 shows a schematic of an interferometer for detecting PFAS. The device combines two very sensitive methods, waveguiding and interferometry, to form waveguide interferometry technology used in rapid low-level detection sensing applications. In optical interferometry, small changes in refractive index are measured along the pathway of an optical beam of light during its propagation. As the light propagates, it generates an optical electromagnetic field, a small fraction of which-known as the evanescent field-enables detection by interacting with the changes in refractive index occurring on the waveguide cover layer. As shown in FIG. 13, an antibody that binds with PFAS is coupled to the waveguide surface. As PFAS is introduced to the functionalized waveguide, PFAS binding to the antibody displaces the sample solution near the waveguide surface, changing the light beam's velocity. Simultaneously, an adjacent reference beam is left unperturbed and is optically combined with the sensing beam to measure the velocity change. This creates an interference pattern that shifts as the refractive index changes, producing a corresponding change in the relative phase measurement.

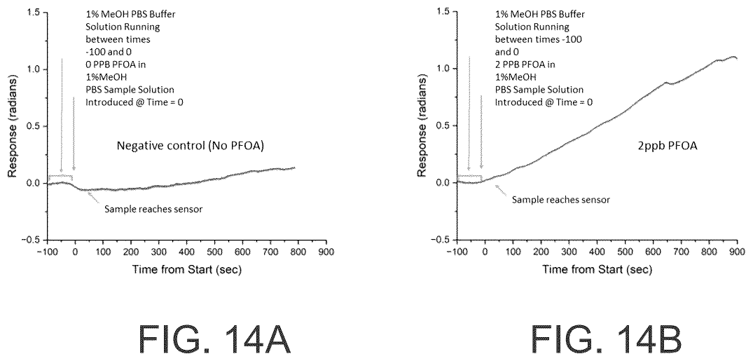

Binding of PFAS binding proteins to PFAS was tested using interferometry. Specifically, binding of anti-PFOA antibody mAB5 to PFOA was measured by comparing the interferometry signal for a 1% methanol PBS buffer containing either 0 ppb PFOA (negative control; FIG. 14A) or 2 ppb PFOA (test sample; FIG. 14B). The control or test solution was run between times −100 and 0, where the sample reached the sensor at time 0. This experiment was done in a clean matrix so that there were no interfering compounds in the water that could distort the signal.

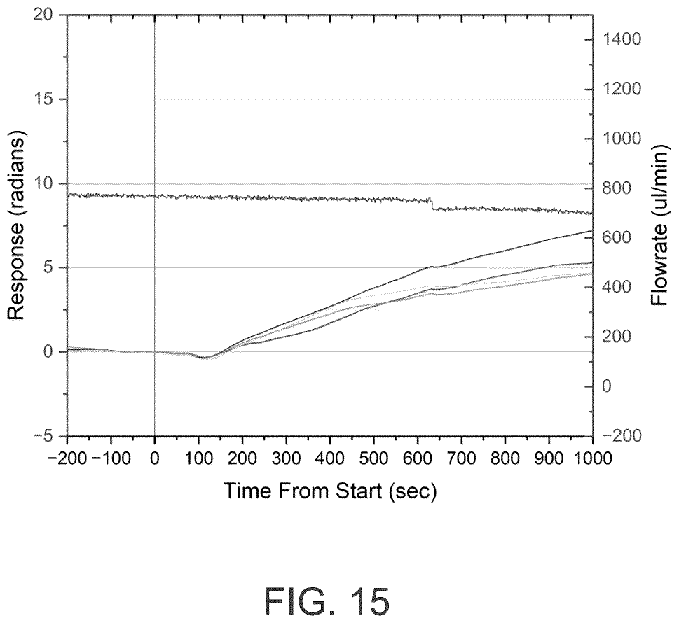

The ability of the mAB5-based interferometry system to detect PFOA in groundwater was also tested. FIG. 15 shows the results of mAB5 binding to PFAS in groundwater containing approximately 7.5 ppb of PFOA. This was collected from a contaminated site and a sample was run to test the detection capabilities of the protein in matrix that could have other interfering compounds. The signal was consistent and approximately proportional to the concentration of PFOA expected in the samples. This is indicated by the signal observed in the matrix and comparing it to signal observed in FIG. 14B, which shows the results of detecting 2 ppb PFOA. The results demonstrate that PFAS binding proteins implemented in interferometry systems can detect PFAS in aqueous samples.

What is claimed is:

1. A composition comprising a per-and polyfluoroalkyl compound (PFAS) binding protein, wherein the PFAS binding protein binds to one or more PFAS compounds.

2. The composition of claim 1 wherein the PFAS binding protein comprises an anti-PFAS antibody, a biosimilar thereof, or an antigen binding fragment of the foregoing.

3. The composition of claim 2, wherein the anti-PFAS antibody is a heterotetrameric antibody or a single domain antibody.

4. The composition of claim 3, wherein the anti-PFAS antibody is heterotetrameric.

5. The composition of claim 4, wherein the heterotetrameric antibody is produced in a chicken, mouse, rat, rabbit, guinea pig, sheep, pig, goat, or horse.

6. The composition of claim 4, wherein the heterotetrameric antibody is polyclonal or monoclonal.

7. The composition of claim 3, wherein the anti-PFAS antibody is a single domain antibody.

8. The composition of claim 7, wherein the single domain antibody is a nanobody or single chain variable fragment (scFv).

9. The composition of claim 8, wherein the single domain antibody is a nanobody.

10. The composition of claim 9, wherein the nanobody is produced in a camelid.

11. The composition of claim 10, wherein the camelid is an alpaca, llama, or camel.

12. The composition of claim 3, wherein anti-PFAS antibody comprises a heavy chain variable region comprising the sequence set forth in one of SEQ ID NOs: 15-19, and a light chain variable region comprising the sequence set forth in one of SEQ ID NOs: 20-24.

13. The composition of claim 12, wherein the heavy chain variable region comprises the sequence set forth in SEQ ID NO: 15 and the light chain variable region comprises the sequence set forth in SEQ ID NO: 20.

14. The composition of claim 12, wherein the heavy chain variable region comprises the sequence set forth in SEQ ID NO: 16 and the light chain variable region comprises the sequence set forth in SEQ ID NO: 21.

15. The composition of claim 12, wherein the heavy chain variable region comprises the sequence set forth in SEQ ID NO: 17 and the light chain variable region comprises the sequence set forth in SEQ ID NO: 22.

16. The composition of claim 12, wherein the heavy chain variable region comprises the sequence set forth in SEQ ID NO: 18 and the light chain variable region comprises the sequence set forth in SEQ ID NO: 23.

17. The composition of claim 12, wherein the heavy chain variable region comprises the sequence set forth in SEQ ID NO: 19 and the light chain variable region comprises the sequence set forth in SEQ ID NO: 24.

18. The composition of claim 12, wherein the antibody comprises mAB1, mAB2, mAB3, mAB4, mAB5, or a biosimilar thereof.

19. The composition of claim 18 wherein the antibody comprises mAB3 or a biosimilar thereof.

20. The composition of claim 18 wherein the antibody comprises mAB5 or a biosimilar thereof.

21. The composition of any one of claims 1-20, wherein the PFAS binding protein is bound to at least one of the PFAS compounds.

22. The composition of claim 1, wherein the PFAS compounds comprise straight chain perfluorinated carboxylic-or sulfonic acid molecules comprising a chain of 4 to 10 carbons in length.

23. The composition of claim 22, wherein the PFAS compounds comprise at least one compound selected from the group consisting of: perfluorobutanoic acid (PFBA), perfluoropentanoic acid (PFPeA), perfluorohexanoic acid (PFHxA), perfluoroheptanoic acid (PFHpA), perfluorooctanoic acid (PFOA), perfluorononanoic acid (PFNA), perfluorodecanoic acid (PFDA), perfluorobutane sulfonate (PFBS), perfluoropentane sulfonic acid (PFPeS), perfluorohexanesulphonic acid (PFHxS), perfluoroheptanesulfonic acid (PfHpS), perfluorooctane sulfonic acid (PFOS), perfluorononanesulfonic acid (PFNS), and perfluorodecane sulfonic acid (PFDS).

24. The composition of claim 23, wherein the PFAS compounds comprise one or more of PFBA, PFPeA, PFHxA, PFHpA, PFOA, PFNA, and PFDA.

25. The composition of claim 24, wherein the PFAS compounds comprise PFOA.

26. A PFAS detection system comprising the composition of any one of claims 1-25, wherein the PFAS binding protein is in a solution or attached to a solid substrate.

27. The PFAS detection system of claim 26, wherein the PFAS binding protein is attached to a solid substrate.

28. The PFAS detection system of claim 27, wherein a cartridge comprises the solid substrate.

29. The PFAS detection system of claim 27 or 28, wherein the solid substrate comprises an electroactive surface.

30. The PFAS detection system of claim 27 or 28, wherein the solid substrate comprises a metal.

31. The PFAS detection system of claim 30, wherein the metal comprises gold.

32. A method of detecting a PFAS compound in a sample, comprising:
(a) contacting the sample with the composition of any one of claim 1-20 or 22-25, or the PFAS detection system of any one of claims 26-31 under conditions that allow the PFAS binding protein to bind the PFAS compound to result in a PFAS binding protein-PFAS compound conjugate; and
(b) detecting the level of the PFAS binding protein-PFAS compound conjugate,
wherein the level of PFAS binding protein-PFAS compound conjugate is indicative of the level of the PFAS compound in the sample.

33. The method of claim 32, wherein the PFAS binding protein is the anti-PFAS antibody of any one of claims 7-11 or a biosimilar thereof.

34. The method of claim 33, wherein the anti-PFAS antibody comprises a heavy chain variable region comprising the sequence set forth in SEQ ID NO: 17 and a light chain variable region comprising the sequence set forth in SEQ ID NO: 22.

35. The method of claim 33, wherein the anti-PFAS antibody comprises a heavy chain variable region comprising the sequence set forth in SEQ ID NO: 19 and a light chain variable region comprising the sequence set forth in SEQ ID NO: 24.

36. The method of any one of claims 32-35, wherein the detecting is by ELISA.

37. The method of any one of claims 32-35, wherein the detecting is by an electrochemical method.

38. The method of any one of claims 32-37, wherein the limit of detection of the method is no greater than single digit parts per trillion.

39. The method of any one of claims 32-38, wherein the sample is a water sample.

40. The method of any one of claims 32-39, comprising pre-treating the water sample by concentrating the PFAS compound before step (a).

41. The method of claim 40, comprising pre-treating the water sample by removing inhibitors of the binding of the PFAS binding protein and the PFAS compound before step (a).

42. A method of detecting a PFAS compound in a sample, comprising:
(a) contacting the sample with the composition of any one of claim 1-20 or 22-25, or the PFAS detection system of any one of claims 26-31, wherein the PFAS binding protein is bound to an electroactive surface to create a PFAS binding protein-bound base, and wherein the contacting is under conditions that allow the PFAS binding protein to bind the PFAS compound to result in a PFAS binding protein-PFAS compound conjugate;
(b) measuring electrochemical signals with a transducer before and after binding of the PFAS compound to the PFAS binding protein-bound base and calculating a difference between the electrochemical signals,
wherein a difference between the electrochemical signals before and after binding of the PFAS compound to the PFAS binding protein-bound base correlates to the amount of the PFAS compound in the sample.

43. The method of claim 42, wherein the sample is a water sample.

44. The method of claim 42 or 43, wherein the PFAS binding protein is the anti-PFAS antibody of any one of claims 13-17 or a biosimilar thereof.

45. The method of claim 44, wherein the anti-PFAS antibody comprises a heavy chain variable region comprising the sequence set forth in SEQ ID NO: 17 and a light chain variable region comprising the sequence set forth in SEQ ID NO: 22.

46. The method of claim 44, wherein the anti-PFAS antibody comprises a heavy chain variable region comprising the sequence set forth in SEQ ID NO: 19 and a light chain variable region comprising the sequence set forth in SEQ ID NO: 24.

47. A method of detecting a PFAS compound in a sample, comprising:
(a) contacting the sample with the composition of any one of claim 1-20 or 22-25, wherein the PFAS binding protein is bound to a surface of an optical planar waveguide, under conditions suitable for the PFAS compound to bind to the PFAS binding protein to generate a PFAS compound-PFAS binding protein conjugate;
(b) exposing the waveguide comprising the PFAS compound-PFAS binding protein conjugate to a sensing beam of polarized light;
(c) optically combining the sensing beam with an adjacent reference beam of polarized light to generate an interference pattern, wherein the interference pattern is indicative of a change in a speed of the sensing beam,
wherein the degree of change in the speed of the sensing beam is indicative of the amount of the PFAS compound in the sample.

48. The method of claim 47, wherein the sample is a water sample.

49. The method of claim 47 or 48, wherein the PFAS binding protein is the anti-PFAS antibody of any one of claims 13-17 or a biosimilar thereof.

50. The method of claim 49, wherein the anti-PFAS antibody comprises a heavy chain variable region comprising the sequence set forth in SEQ ID NO: 17 and a light chain variable region comprising the sequence set forth in SEQ ID NO: 22.

51. The method of claim 49, wherein the anti-PFAS antibody comprises a heavy chain variable region comprising the sequence set forth in SEQ ID NO: 19 and a light chain variable region comprising the sequence set forth in SEQ ID NO: 24.

52. The method of any one of claims 32-51, wherein the PFAS compound in the sample comprises one or more straight chain perfluorinated carboxylic-or sulfonic acid molecules comprising a chain of 4 to 10 carbons in length.

53. The method of claim 52, wherein the PFAS compound in the sample comprises at least one compound selected from the group consisting of: perfluorobutanoic acid (PFBA), perfluoropentanoic acid (PFPeA), perfluorohexanoic acid (PFHxA), perfluoroheptanoic acid (PFHpA), perfluorooctanoic acid (PFOA), perfluorononanoic acid (PFNA), perfluorodecanoic acid (PFDA), perfluorobutane sulfonate (PFBS), perfluoropentane sulfonic acid (PFPeS), perfluorohexanesulphonic acid (PFHxS), perfluoroheptanesulfonic acid (PfHpS), perfluorooctane sulfonic acid (PFOS), perfluorononanesulfonic acid (PFNS), and perfluorodecane sulfonic acid (PFDS).

54. The method of claim 53, wherein the PFAS compound in the sample comprises one or more of PFBA, PFPeA, PFHxA, PFHpA, PFOA, PFNA, and PFDA.

55. The method of claim 54, wherein the PFAS compound in the sample comprises one or more of PFOA, PFOS, PFHxS, and PFBA.

56. The method of claim 55, wherein the PFAS compound in the sample comprises PFOA.


SEQUENCE LISTING (24 sequences)

SEQ ID NO: 1 - DNA, 408 bp, Oryctolagus cuniculus
  source 1..408: mol_type = other DNA
atggagactgggctgcgctggcttctcctggtcgctgtgctcaaaggtgtccagtgtcagtcggtgaaggagtccgagggaggtctcttcaagccaacggataccctgacactcacctgcacagcctctggattctccctcagtagctatggagtgagctgggtccgccaggctccagggaacgggctggaatggatcggatccatagaggatggtggtagcgcatactacgcgagctgggcgaaaagccgatccaccatcactagcaacaccaacctgaatacggtgactctgcaaatgaccagtctgacagccgcggacacggccacctatttctgtgggagtcttagtattggtagtgcttatcctggcatctggggcccaggcaccctggtcaccgtctcctcg

SEQ ID NO: 2 - DNA, 399 bp, Oryctolagus cuniculus
  source 1..399: mol_type = other DNA
atggacacgagggcccccactcagctgctggggctcctgctgctctggctcccaggtgccacatttgccatcgtgatgacccagactccatcttccaagtctgtccctgtgggagacacagtcaccatcaaatgccaggccagtcagagcattggtactaggttagcctggtttcagcagaaaccagggcaacctcccaagcgcctgatctattctgcatccactctggcatctggggtcccatcgcggttcagcggcagtggatctgggacacagttcactctcaccatcagcgatgtggtgtgtgacgatgctgccgcttactactgtggaggatataaaagtagtagtagtgatggtatcgctttcggcggagggaccgaggtggtggtcaaaggt

SEQ ID NO: 3 - DNA, 414 bp, Oryctolagus cuniculus
  source 1..414: mol_type = other DNA
atggagactgggctgcgctggcttctcctggtcgctgtgctcaaaggtgtccagtgtcagtcggtgaaggagtccgagggaggtctcttcaagccaacggataacctgacactcacctgcgcagtctctagattctccctcagtagctatggagtgagctgggtccgccaggctccagggaacgggctggaatggatcggatacatttatactgatggtagcgcatactacgcgagctgggcgaaaagccgatccaccatcaccagaaacaccaacctgaacacggtgactctgaaaatgaccagtttgacagccgcggacacggccacctatttctgtgcgagacatagtagtggctggggaattgactactttaacgtctggggcccaggcaccctggtcaccgtctcctca

SEQ ID NO: 4 - DNA, 399 bp, Oryctolagus cuniculus
  source 1..399: mol_type = other DNA
atggacacgagggcccccactcagctgctggggctcctgctgctctggctcccaggtgccacatttgccgccgtgctgacccagactccatcctccgtgtctgcagctgtgggaggcacagtcaccatcaattgccaggccagtcagagtgtttataataacaactacttatcctggtatcagcagaaaccagggcagcctcccaagctcctgatctatctagcatctactctggcatctggggtcccatcgcggttcagtggcagtggatctgggacagagttcactctcaccatcagcgacctggagtgtgacgatggtgccacttactactgtgcaggcggtgttaccagtagtgatagacctttcggcggagggaccgaggtggtggtcaaaggt

SEQ ID NO: 5 - DNA, 405 bp, Oryctolagus cuniculus
  source 1..405: mol_type = other DNA
atggagactgggctgcgctggcttctcctggtcgctgtgctcaaaggtgtccagtgtcagtcgctggaggagtccgagggaggtctcttcaagcctggaggaaccctgacactcacctgcacagtctctggattttccctcaatatttattcaataagttgggtccgacaggctccagggaaggagctggaatggatcggaaccatgtctaatgtcgatgccacatactacgcgaactgggcgaaaagccggtccaccattaccagaaacatcaatgagagcacagtgactctgaaaatgcccagtctgacagtcgcggacacggccacgtatttctgtgccagatatatctatggtgctggatatgatatttggggcccaggcaccctggtcaccgtctcctca

SEQ ID NO: 6 - DNA, 408 bp, Oryctolagus cuniculus
  source 1..408: mol_type = other DNA
atggacacgagggcccccactcagctgctggggctcctactgctctggctcccaggtgccagatgtgctgacattgtgatgacccagactccagcctccgtggaggcagctgtgggaggcacagtcaccatcaagtgccaggccagtcagagcattagtagttggttatcctggtatcagcagaaaccagggcagcctcccaagctcctgatctacaaggcatccactctggcatctggggtcccatcgcggttcaaaggcagtggatctgggacagagtacactctcaccatcagcggcgtgcagtgtgccgatgctgccacttactactgtcaaagtgtttattatagtagtagtactaattatgggaatactttcggcggagggaccgaggtggtggtcaaaggt

SEQ ID NO: 7 - DNA, 411 bp, Oryctolagus cuniculus
  source 1..411: mol_type = other DNA
atggagactgggctgcgctggcttctcctggtcgctgtgctcaaaggtgtccagtgtcaggagcagctggaggagtccaggggaggccttatcaagccaacggataccctgacactcacctgcacagcctctggattctccctcagcaattatggaataaactgggtccgccaggctccaggggaggggctggaatggatcggagccattcgtggtagtggcttcatatactacgcgacctgggcgaaaagccgatccaccatcaccagaaacaccaacctgaacacggtgactctgaaaatgaccagtctgacagccgcggacacggccacctatttctgtgcgagaagggatgggttatattctgccatggacccctggggcccagggaccctcgtcaccgtctcttca

SEQ ID NO: 8 - DNA, 396 bp, Oryctolagus cuniculus
  source 1..396: mol_type = other DNA
atggacacgagggcccccactcagctgctggggctcctgctgctctggctcccaggtgccatatgtgaccctgtgctgacccagactccatcttccacgtctgcagctgtgggaggcacagtcaccatcagttgccagtccagtgacagtgtttataagaacaactacttagcctggtttcagcagaaaccagggcagcctcccaaggtcttgatctaccatgcatccaaactggcatctggggtcccatcgcggttcagtggcagtggatctgggacacagttcactctcaccatcagcggcgtacagtgcgacgatgctgccacttactactgtctaggagaatatagtgatacgcatgttttcggcggagggaccgaggtggtcgtcaaaggt

SEQ ID NO: 9 - DNA, 417 bp, Oryctolagus cuniculus
  source 1..417: mol_type = other DNA
atggagactgggctgcgctggcttctcctggtcgctgtgctcaaaggtgtccagtgtcagtcggtgaaggagtccgagggaggtctcttcaaaccaacagataccctgacactcacctgcacagcctccggattctccctcagtagttatggagtgatctgggtccgccaggctccaggaaaggggctgcaatggatcggattcattaatatctatggtaccccaagctacgcgagctgggcgatgggccgctccaccatcaccggaaacaccaatctgaacacggtgattctgaaaatgaccagtctgacagtcgcggacacggccacctatttctgtgcgagctcggtgggtagtgatggttatctctattcttctgacatctggggcccaggcaccctagtcaccgtctcctca

SEQ ID NO: 10 - DNA, 396 bp, Oryctolagus cuniculus
  source 1..396: mol_type = other DNA
atggacacgagggcccccactcagctgctggggctcctgctgctctggctcccaggtgccatatgtgaccctgtgctgacccagactccatcctccgtgtctgcagctgtgggaggcacagtcaccatcaattgccaggccagtcagagtgtttataataataaatgtttagcctggtatcagcagaaaccagggcagcctcccaagctcctgatctatggtgcatccactctggcatctggggtcccatcgcggttcagtggcagtggatctgggacacagttcactctcaccatcagcgacctggagtgtgacgatgctgccacttattactgtgcaggcggttattatagtgaaattcatttcggcggagggaccgaggtggtggtcaagggt

SEQ ID NO: 11 - protein, 135 aa, Oryctolagus cuniculus
METGLRWLLLVAVLKGVQCQSLEESEGGLFKPGGTLTLTCTVSGFSLNIYSISWVRQAPGKELEWIGTMSNVDATYYANWAKSRSTITRNINESTVTLKMPSLTVADTATYFCARYIYGAGYDIWGPGTLVTVSS

SEQ ID NO: 12 - protein, 136 aa, Oryctolagus cuniculus
MDTRAPTQLLGLLLLWLPGARCADIVMTQTPASVEAAVGGTVTIKCQASQSISSWLSWYQQKPGQPPKLLIYKASTLASGVPSRFKGSGSGTEYTLTISGVQCADAATYYCQSVYYSSSTNYGNTFGGGTEVVVKG

SEQ ID NO: 13 - protein, 139 aa, Oryctolagus cuniculus
METGLRWLLLVAVLKGVQCQSVKESEGGLFKPTDTLTLTCTASGFSLSSYGVIWVRQAPGKGLQWIGFINIYGTPSYASWAMGRSTITGNTNLNTVILKMTSLTVADTATYFCASSVGSDGYLYSSDIWGPGTLVTVSS

SEQ ID NO: 14 - protein, 132 aa, Oryctolagus cuniculus
MDTRAPTQLLGLLLLWLPGAICDPVLTQTPSSVSAAVGGTVTINCQASQSVYNNKCLAWYQQKPGQPPKLLIYGASTLASGVPSRFSGSGSGTQFTLTISDLECDDAATYYCAGGYYSEIHFGGGTEVVVKG

SEQ ID NO: 15 - protein, 117 aa, Oryctolagus cuniculus
QSVKESEGGLFKPTDTLTLTCTASGFSLSSYGVSWVRQAPGNGLEWIGSIEDGGSAYYASWAKSRSTITSNTNLNTVTLQMTSLTAADTATYFCGSLSIGSAYPGIWGPGTLVTVSS

SEQ ID NO: 16 - protein, 119 aa, Oryctolagus cuniculus
QSVKESEGGLFKPTDNLTLTCAVSRFSLSSYGVSWVRQAPGNGLEWIGYIYTDGSAYYASWAKSRSTITRNTNLNTVTLKMTSLTAADTATYFCARHSSGWGIDYFNVWGPGTLVTVSS

SEQ ID NO: 17 - protein, 116 aa, Oryctolagus cuniculus
QSLEESEGGLFKPGGTLTLTCTVSGFSLNIYSISWVRQAPGKELEWIGTMSNVDATYYANWAKSRSTITRNINESTVTLKMPSLTVADTATYFCARYIYGAGYDIWGPGTLVTVSS

SEQ ID NO: 18 - protein, 118 aa, Oryctolagus cuniculus
QEQLEESRGGLIKPTDTLTLTCTASGFSLSNYGINWVRQAPGEGLEWIGAIRGSGFIYYATWAKSRSTITRNTNLNTVTLKMTSLTAADTATYFCARRDGLYSAMDPWGPGTLVTVSS

SEQ ID NO: 19 - protein, 120 aa, Oryctolagus cuniculus
QSVKESEGGLFKPTDTLTLTCTASGFSLSSYGVIWVRQAPGKGLQWIGFINIYGTPSYASWAMGRSTITGNTNLNTVILKMTSLTVADTATYFCASSVGSDGYLYSSDIWGPGTLVTVSS

SEQ ID NO: 20 - protein, 111 aa, Oryctolagus cuniculus
AIVMTQTPSSKSVPVGDTVTIKCQASQSIGTRLAWFQQKPGQPPKRLIYSASTLASGVPSRFSGSGSGTQFTLTISDVVCDDAAAYYCGGYKSSSSDGIAFGGGTEVVVKG

SEQ ID NO: 21 - protein, 111 aa, Oryctolagus cuniculus
AAVLTQTPSSVSAAVGGTVTINCQASQSVYNNNYLSWYQQKPGQPPKLLIYLASTLASGVPSRFSGSGSGTEFTLTISDLECDDGATYYCAGGVTSSDRPFGGGTEVVVKG

SEQ ID NO: 22 - protein, 114 aa, Oryctolagus cuniculus
ADIVMTQTPASVEAAVGGTVTIKCQASQSISSWLSWYQQKPGQPPKLLIYKASTLASGVPSRFKGSGSGTEYTLTISGVQCADAATYYCQSVYYSSSTNYGNTFGGGTEVVVKG

SEQ ID NO: 23 - protein, 110 aa, Oryctolagus cuniculus
DPVLTQTPSSTSAAVGGTVTISCQSSDSVYKNNYLAWFQQKPGQPPKVLIYHASKLASGVPSRFSGSGSGTQFTLTISGVQCDDAATYYCLGEYSDTHVFGGGTEVVVKG

SEQ ID NO: 24 - protein, 110 aa, Oryctolagus cuniculus
DPVLTQTPSSVSAAVGGTVTINCQASQSVYNNKCLAWYQQKPGQPPKLLIYGASTLASGVPSRFSGSGSGTQFTLTISDLECDDAATYYCAGGYYSEIHFGGGTEVVVKG